(19) United States
(12) Patent Application Publication
(10) Pub. No.: US 2010/0227393 A1
(43) Pub. Date: Sep. 9, 2010

(54) LIVER STEM CELLS: ISOLATION OF HEPATIC PROGENITOR CELLS FROM THE HUMAN GALL BLADDER

(76) Inventor: Eric Lagasse, Pittsburgh, PA (US)

Correspondence Address: Hirshman Law, LLC, Gatehouse Building, 101 W. Station Square Dr., Suite 500, PITTSBURGH, PA, 15219, US

(21) Appl. No.: 12/718,579

(22) Filed: Mar. 5, 2010

Related U.S. Application Data
(60) Provisional application No. 61/209,452, filed on Mar. 6, 2009.

(51) Int. Cl. C12N 5/07

(52) U.S. Cl. 435/325

(57) ABSTRACT

Methods of preparing multipotent cell populations from gallbladder are provided. Also provided are enriched multipotent cell populations from gallbladder and methods of differentiating the multipotent cell populations to prepare, for example, hepatocytes.

This application claims the benefit under 35 U.S.C. §119(e) to U.S. Provisional Patent Application No. 61/209,452, filed Mar. 6, 2009, which is incorporated herein by reference in its entirety.

Identification of multipotential progenitor populations in mammalian tissues is important both for therapeutic potential and an understanding of development and physiology. Progenitor populations are potentially useful ideal targets for cell transplantation, regenerative medicine and gene or cellular therapy.

Liver disease is the eighth leading cause of mortality in the US. However, current treatments remain limited in feasibility. Currently over 17,000 people are on the waiting list for a liver transplant, many with chronic liver injury. However, only a third undergoes a transplant. Transplantation of mature hepatocytes is being explored as an alternative to orthotopic liver transplantation. Unfortunately, the number donor livers for isolation of hepatocytes, is limited. Furthermore, because transplantation of hepatocytes is limited by their size and fragility, alternative sources for long-term engraftment are being explored as cell-based therapies. One such source is the liver stem/progenitor cell.

Bipotent cells (termed hepatoblasts or “oval cells” as identified in an animal model of liver injury) capable of self-renewal and differentiation into hepatic and bile duct lineages have been identified in rodent models (Kubota H, and Reid L M. 2000. PNAS 97 (22): 12132-12137; Tanimizu N, Nishikawa M et al. 2003. J Cell Sci. 116 (Pt. 9): 1775-1786; Nierhoff D, Ogawa A et al. 2005. Hepatology. 42 (1):130-139; Suzuki A, Zheng Y W et al. 2002. J Cell Biol 156 (1): 173-184). Identification of such cells in humans has proved more difficult, though recent literature indicates the presence of hepatoblasts and hepatic stem cells in the human fetal liver (Schmelzer E, Wauthier E et al. 2006. Stem Cells. 24 (8): 1852-1858; Dan Y Y, Riehle K J et al. 2006. PNAS. 103(26): 9912-9917). Oval cells emerge from the terminal branches of the intrahepatic bile ducts, called the Canals of Hering (Dabeva M D, Shafritz D A. 1993. Am J. Pathol. 143: 1606-1620; Sarrafi C, Lalani E N et al. 1994. Am J. Pathol. 145: 1243-1253; Theise N D, Saxena R et al. 1999. Hepatology. 30: 1425-1433). Work in adult human liver indicates that cells with a similar phenotye in vitro are derived from the intra-hepatic bile duct. Some studies explore the possibility of the foregoing identification of progenitor in the mouse extra-hepatic bile duct. Furthermore, it is know that the entire hepatic system, including the gall bladder, develops from the ventral foregut endoderm. Given this close genealogical relationship and that hepatic progenitor cells appear associated with the intra-hepatic biliary system, these results suggest that a putative progenitor population might exist in the human gall bladder.

### SUMMARY

The isolation and characterization of multipotent cells from adult and fetal human gall bladder is described herein. Cells from adult and fetal gall bladder were successfully expanded in vitro. Cultures exhibit morphological heterogeneity, from immature epithelial to mature cells, ranging from undifferentiated colonies of cells to organized glandular formations. Pleiomorphic cultures of fetal and adult gall bladder cells are phenotypically similar, indicating a possible expansion of a related primitive cell population. Flow cytometric profiles are comparable between the adult and the fetal: a double positive population EpCAM⁺, CD26^{low} with a small percentage expressing a novel cell surface marker called gall bladder marker (GbM). GbM is also known as CD227 or MUC-1. A limiting dilution analysis performed on one sample shows that the frequency of colony forming cells in the GbM subpopulation was enriched by a seven-fold over the GbM⁻ population. These preliminary results indicate the presence of progenitor cells in fetal and adult gall bladder that can be prospectively isolated. These newly described progenitor cells from human gall bladder represent good candidates to test hepatic differentiation.

### BRIEF SUMMARY OF THE DRAWINGS

FIG. 1 shows the expansion of gall bladder cells in vitro for Example 1.

FIG. 2 shows the expression of GbM, CK19, and EpCAM in human gall bladder.

FIG. 3 shows flow cytometric analyses of human gall bladder populations.

FIG. 4 shows the CFU frequency in GbM sorted cell populations.

FIG. 5 shows representative light micrographs of human and GFP⁺ mouse adult gallbladder grown on irradiated feeder cells.

FIGS. 6A and 6B provides flow cytometric profiles of EpCAM in human and mouse adult gallbladder cells, respectively.

FIG. 7 shows Limiting Dilution Analysis of human and mouse gallbladder cells from primary tissue and in culture on feeder cells.

FIG. 8A is a phenotypic profile of Clone A4 compared to the bulk population. FIG. 8B provides photomicrographs showing clone A4 consisted of small-cell colonies (left panel), ductular structures (middle panel), and 3D organotypic structures (right panel) in culture. Black Scale bars=50 μm, White Scale bar=100 μm

FIG. 9. Formation of ductular structures in vivo.

FIG. 10 shows an RT-PCR experiment indicating up-regulation of albumin gene expression is seen in mouse gallbladder cells treated with HGF/EGF/Dex.

FIG. 11A shows that human gallbladder cells form two distinct type of structures: gallbladder-like structures with narrow lumen that are attached to the dish surface and cyst-like structures suspended in the matrigel. FIG. 11B: (i) shows mouse gallbladder cells exhibit similar morphogenesis, and (ii) shows slice in a confocal stack of a cyst depicting a narrow lining of cells that surrounds a large lumen.

### DETAILED DESCRIPTION

All ranges or numerical values stated herein, whether or not preceded by the term “about” unless stated otherwise are considered to be preceded by the term “about” to account for variations in precision of measurement and functionally equivalent ranges.

As used herein, the terms “comprising,” “comprise” or “comprised,” and variations thereof, are meant to be open ended. The terms “a” and “an” are intended to refer to one or more.

As used herein, the term “patient” or “subject” refers to members of the animal kingdom including but not limited to human beings.

The focus of the work described herein is to isolate and characterize an organ-specific steady-state stem cell population in the gallbladder and to define this population's plasticity, e.g., towards the hepatocyte lineage.

According to one embodiment of the present invention, a method of isolating progenitor cells (e.g., a stem cell population) is provided. By progenitor cells, it is meant cells that can differentiate or mature into one or more cell types, such as hepatocytes and beta cells. These cells may include multipotent cells, pluripotent cells or stem cells. The method comprises: dissociating gallbladder cells; enriching the gallbladder cells for cells that are EpCAM+, and optionally are GbM+, CD133+, CD49f+, and/or CD13−, for example CD133+, CD49f+, and CD13−; optionally enriching the progenitor cell population by growth in culture in vitro culture, thereby producing an enriched population of stem cells. By “enriched” in the context of the progenitor cell populations described herein, it is meant that the percentage of cells exhibiting a specific phenotype or genotype is increased in a cell population beyond the percentage found in normal tissue Likewise, the term “enriching” refers to a process for producing a cell population enriched for a specific phenotype or genotype. Thus, a “method of producing an enriched progenitor cell population” and like terms involves increasing the percentage of progenitor cells in a population of cells above what is found naturally, for instance in a population of cells obtained from a tissue by dissociating the cells and doing nothing further.

The cells are dissociated from gallbladder tissue in any useful manner. A most common method is to degrade the tissue, for example the extracellular matrix of the tissue, so that cells can be dissociated from the tissue. In one non-limiting example, human gallbladder is chopped, minced or otherwise comminuted, and then digested with one or more proteinases, such as, without limitation: trypsin, pepsin, and collagenase. The tissue may be digested multiple times or for any duration that enables the cells to be dissociated from or the tissue sample.

Once the cells are dissociated, they optionally may be cultured, to maintain or expand the number of cells capable of growing in culture. Because stem cells can renew themselves, often faster than other cell types, the process of culturing the cells in vitro may enrich the population of cells for stem cells. As indicated in the examples below, the cells may be cultured on a feeder layer of cells. This is not considered to be necessary in many instances, given the myriad of cell culture methods described and/or commercially available; including, growth on plates, on or in secreted substances such as matrigel, decellularized extracellular matrix preparations, natural or synthetic polymeric cell growth scaffolds, cell reactors, etc. The cells are cultured in any useful cell growth medium either comprising serum supplementation, such as fetal bovine serum, or which is serum-free. A useful example of such medium is DMEM/F12 (Fisher 10-090-CV)+0.5% FBS+1% Insulin, Transferrin, Selenium Supplement ITS) (Fisher#25-080-CR)+25 μg/ml Gentamycin (Sigma#G1397). The cells may be cultured in an incubator at 37° C. with CO₂. Cell populations can be expanded from single cells in progressively larger plates, flasks or other surfaces. Other culture systems may prove useful, such as hollow-fiber open or closed culture systems.

The cells optionally can be cultured on a feeder layer of cells. The cells may be stromal (e.g., fibroblasts), but cells of epithelial in origin may be preferred in many instances. The cells can be from any source so long as the growth of the feeder cells is restricted. The cells are preferably quiescent or their growth is preferably inhibited or restricted in any manner, such as by γ-irradiation. The feeder layer may be a cancer cell line, because in such an instance, it is relatively simple to obtain large or renewable populations of feeder cells. In the examples below, the cell line is a rat breast ductal carcinoma. In order to prevent growth of expansion of the feeder cells, the cells are irradiated prior to use in cell culture as a feeder layer. Adequate radiation doses for γ-irradiation include from 15,000 to 20,000 rads, and approximately 17,000 rads. In any case, determining other conditions at which the cells can be cultured in light of the present disclosure is within the capabilities of those of ordinary skill in the art, and does not require undue experimentation.

To enrich the cells for EpCAM+ cells, any useful separation method may be employed. In one example, the gallbladder cells are enriched by affinity, by binding the cells with an anti-EpCAM binding reagent (e.g., an antibody or fragment thereof), such as an antibody, and physically separating the cell that are bound to the anti-EpCAM binding reagent. These steps can be repeated with or preceded by purifying cells in the same manner by binding to an anti GbM, anti-CD133, anti-CD49f and/or anti-CD13 binding reagents (all of which are commercially available). This can be accomplished by attaching the anti-EpCAM, anti GbM, anti-CD133, anti-CD49f and/or anti-CD13 binding reagents to substrates such as a surfaces of cell culture chambers (e.g., panning) or a bead for an affinity column, or a magnetic bead for magnetic purification. In such an embodiment, the method comprises contacting the dissociated gallbladder cells with the substrate-bound binding reagent, washing unbound cells from the substrate; and eluting bound cells from the substrate. Fluorescence activated cell sorting (FACS) methods, as are broadly known in the relevant arts also may be used to separate the cell populations. In such an embodiment, a fluorescently-labeled binding reagent, such as an antibody, is used to specifically label certain cells. Because cell sorting can be performed using more than one markers simultaneously by labeling the cells with binding reagents that fluoresce at different wavelengths, EpCAM+ and, optionally GbM+, CD133+, CD49f+, CD13-markers may be selected in essentially one, two or three passes, but not requiring re-labeling of the cells between passes.

Once EpCAM+ cells are enriched, the stem cell population may be enriched by culturing in vitro, essentially as described above.

In yet another embodiment, a method of preparing hepatocytes is provided. The method comprises growing EpCAM+ cells (optionally also) isolated from a human gall bladder that grow on an epithelial cell feeder layer. In one embodiment, GbM+, EpCAM+ cells are sandwiched between collagen and matrigel gels and differentiated in the presence of one or more of hepatocyte growth factor (HGF), epidermal growth factor (EGF), and Dexamethasone (Dex).

Also provided is a progenitor cell preparation comprising gallbladder cells enriched for EpCAM+ cells, and optionally GbM+, CD133+, CD49f+, and/or CD13-cells, for example EpCAM+, CD133+, CD49f+, CD13− cells. The enriched progenitor cell population may be further enriched by expanding the enriched cell population in culture, as described above. These cell populations may comprise progenitor cells and non-progenitor cells, but by virtue of the selection for (enrichment for) cells bearing the marker(s), the percentage of progenitor cells is greatly increased.

The term “binding reagent” and like terms, refers to any compound, composition or molecule capable of specifically or substantially specifically (that is with limited cross-reactivity) binding another compound or molecule, which, in the case of immune-recognition contains an epitope. In many instances, the binding reagents are antibodies, such as polyclonal or monoclonal antibodies. “Binding reagents” also include derivatives or analogs of antibodies, including without limitation: Fv fragments; single chain Fv (scFv) fragments; Fab′ fragments; F(ab′)₂ fragments; humanized antibodies and antibody fragments; camelized antibodies and antibody fragments; and multivalent versions of the foregoing. Multivalent binding reagents also may be used, as appropriate, including without limitation: monospecific or bispecific antibodies, such as disulfide stabilized Fv fragments, scFv tandems ((scFv) fragments), diabodies, tribodies or tetrabodies, which typically are covalently linked or otherwise stabilized (i.e., leucine zipper or helix stabilized) scFv fragments. “Binding reagents” also include aptamers, as are described in the art. Binding partners, such as, without limitation, biotin/avidin and receptor/substrate combinations also are considered to be within the class of “binding reagents,” though antibodies and their respective antigens also are considered to be binding partners. Further, two or more binding partners may be included in a single composition (e.g., a polypeptide chain). In one embodiment, this is a string of epitopes, such as the hemagglutinin triplet described below. In such a configuration, the epitopes contained in one compound (e.g., a polypeptide chain) do not have to be identical, as is the case with the HA triplet described herein.

### Example 1

### The Isolation and Analysis of Hepatic Progenitor Cells from the Human Gall Bladder

### Methods

Cells: Adult human gallbladders were treated with repeated rounds of collagenase and trypsin. The isolated cells were plated on rat breast ductal carcinoma cell lines as feeder cells. The growth of the feeder cells was inhibited using γ-irradiation at 17,000 rads. Confluent monolayers were formed by plating the cells at 70,000 cells/mm². Mouse gallbladder cells were isolated from C57BL/6 mice and grown in the same manner. Human and mouse gallbladder cells expanded in vitro on rodent epithelial feeder cells exhibit pleiomorphic growth indicative of heterogeneity in the cell population.
Immuno-histochemistry. EpCAM (mAb: AbCAM) was stained on paraffin section after having performed an antigen retrieval step with sodium citrate.
Flow Cytometry Cell populations were stained with (FITC), CD166-Phycoerythrin (PE), EpCAM-Allophycocyanin (APC), CD49f-PE, CD133.1-APC, CD117-APC, Sca-1-APC-Cy7 and HLA-ABC-APC-Cy7. HLA-ABCs are Class I MHC molecules present on most human cells and are required to separate the human cells from the rodent feeders. Cells were sorted with a BD FACSVantage™ SE Cell Sorter. An Automated Cell Deposition Unit (ACDU) sorts were carried out as indicated into 96-well plates.
Limiting Dilution Analysis: Cell populations in question were sorted into 96-well plates at different cells/well in each row. Colonies were scored after 4-6 weeks of culture and the number of positive wells/row determined. CFU frequency±SE was determined from L-Calc®.
Single Cell Clonogenic Assay: A flask of GFP⁺ mouse gallbladder cells were trypsinized and gfp+ cells were added to a 96-well plate seeded with the rodent epithelial cell layer at 1 cell/well. Colonies were scored after 4-6 weeks in culture and then passaged into larger flasks.
Kidney Injections: GFP⁺ mouse gallbladder cells in culture were injected into the renal subcapsular space of WT mice at 500,000 cells/mouse with a Hamilton syringe. Mice were killed at time intervals of 2 W, 3 W and 4 W.

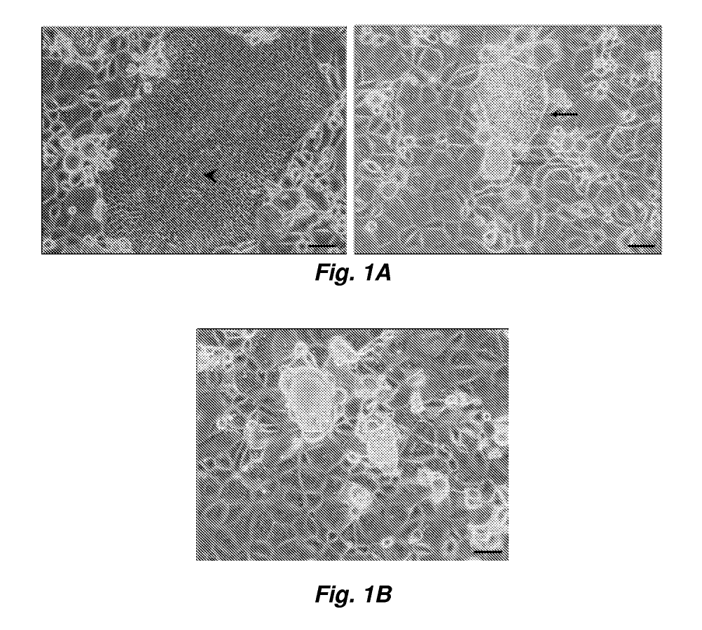

FIG. 1 shows the expansion of gall bladder cells in vitro. Shown are light micrographs of cells from two adult gall bladders and one fetal gall bladder grown on irradiated feeder cells. Light micrographs are shown for HuAL GB1 in FIG. 1A, HuFL GB36 in FIG. 1B, and HuAL GB3 in FIG. 1C. All three cultures exhibited morphological heterogeneity. Smaller cells with large nuclear/cytoplasmic ratios, typical of more primitive cells, can be seen in both HuAL GB1 and HuFL GB36 (arrowheads). More differentiated biliary-type cells can be seen (black arrow) and glandular structures (red arrows) can be seen as well. Scale bars are 50 μm.

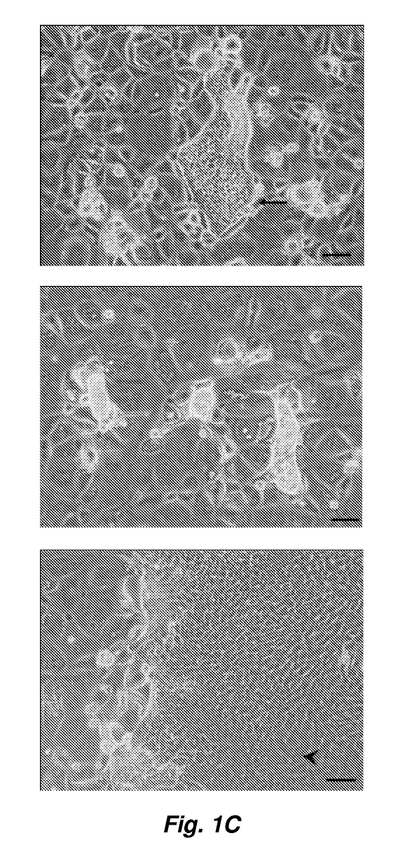

FIG. 2 shows the expression of GbM, CK19, and EpCAM in human gall bladder. FIG. 2A shows a composite light micrograph showing EpCAM expression in HuAL GB1. Expression is seen in mucosal structures (arrowhead) and in the columnar epithelium that lines the lumen (arrow). Frozen sections that were stained with GbM, CK19, EpCAM (Red: Alexa Fluor 594) and counterstained with Hoechst (nuclear) are shown for HuAL GB3 in FIG. 2B and HuFL GB36 in FIG. 2C. GbM was detected with anti-CD227 antibody, also known as anti-MUC1 (available from BD). EpCAM+ cells were seen in mucosal structures, which co-stain for CK19 and GbM. Scale bars are 50 μm.

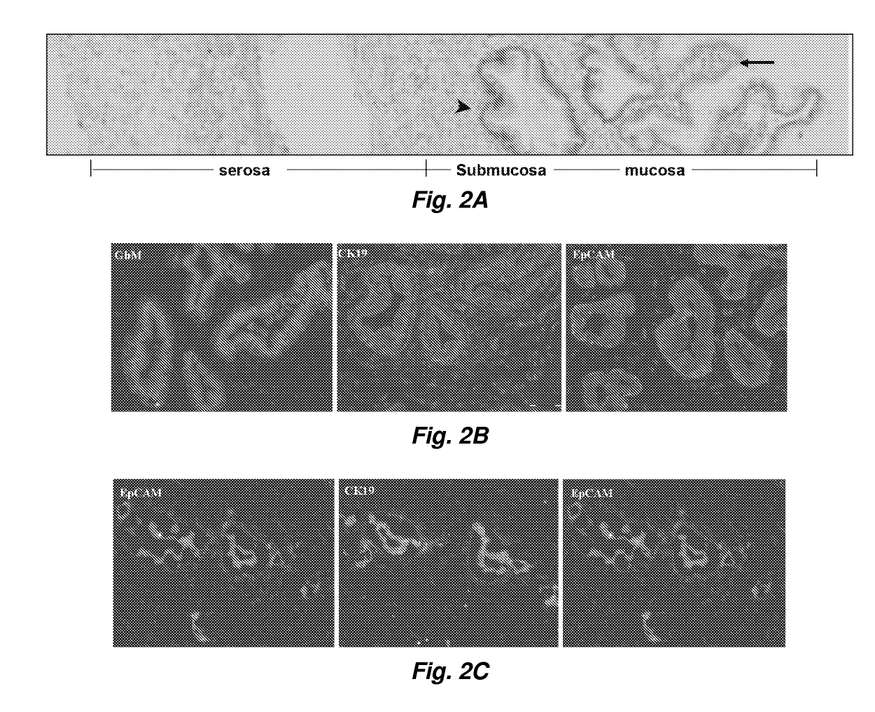

FIG. 3 shows flow cytometric analyses of human gall bladder populations. Gall Bladder Cultures (Passage 0) were stained with EpCAM-APC, CD26-PE (DPPIV), and GbM-FITC. All three cultures were EpCAM⁺, CD26^{-/flow}, with a sub-population being GbM⁺ (approximately 10% and 15% in HuAL GB1 and HuFl GB36 respectively, but 1% in HuAL GB3).

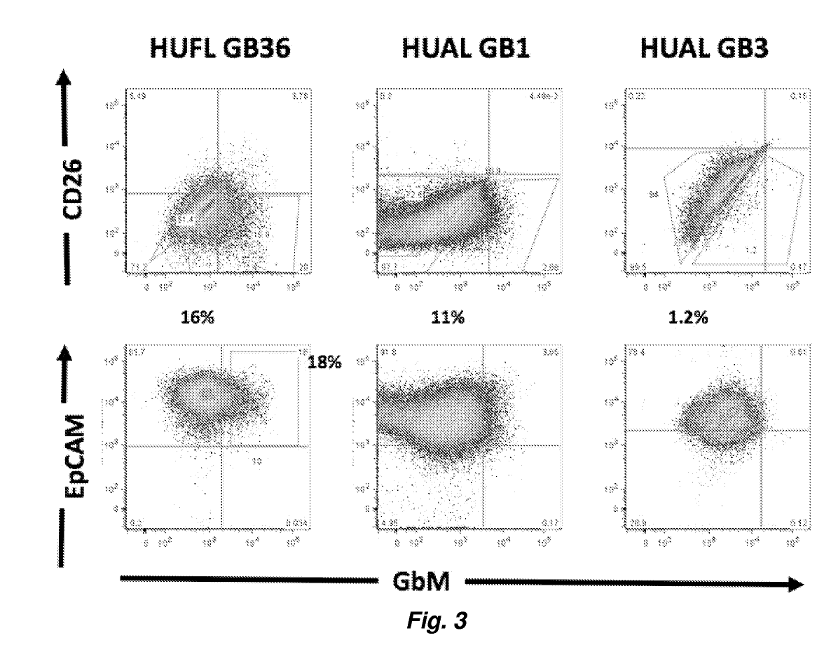

FIG. 4 shows the CFU frequency in GbM sorted cell populations. A Limiting Dilution Analysis (LDA) on GbM⁺, GbM⁻, and HLA-ABC⁺ sorted cells was performed. After three weeks in culture, colonies were counted and the frequency of clonogenic cells determined using L-Calc®. FIG. 4A shows a regression analysis of the GbM⁺ cells versus controls. FIG. 4B shows CFU frequency in HuAL GB1 and HuFL GB36 cultures as determined by L-Calc®.

All three gall bladder cultures expanded in vitro exhibit phenotypic heterogeneity along a continuum from a more primitive morphology to a more differentiated one. Both the columnar epithelium lining the cholecystic lumen and the mucosal epithelium stained for EpCAM (marker of proliferating cells) and CK19 (biliary marker). In addition, a novel stem cell marker, GbM, co-localized with both EpCAM and CK19.

The LDA of HuAL GB1 quantified the increase in CFU frequency that the enrichment for GbM affords. GbM⁺ cells show a seven-fold increase in CFU frequency over the GbM⁻ cells. Inconsistent data with HuFL GB36 imply that the experiment needs to be repeated. Flow cytometric analyses of the gall bladder cells expanded in culture revealed that GbM stains a subpopulation of cells within human gall bladder. The percentage of the GbM⁺ sub-population in HuAL GB1 and HuAL GB3 varied considerably. Therefore, more samples need to be examined to elucidate the significance of this difference.

The percentage of GbM⁺ cells in HuAL GB3 was small. A sufficient number of GbM+ cells could not be sorted for an LDA. However, GbM+ cells were sorted into culture and the CFU frequency was determined to be roughly 1/50 (the GbM⁻ sorted cells did not grow). These data are consistent with the notion that selection for GbM results in an increase in CFU frequency.

### Conclusions

1. All three gall bladder cultures expanded in vitro exhibit phenotypic heterogeneity along a continuum from a more primitive morphology to a more differentiated one.
2. Both the columnar epithelium lining the cholecystic lumen and the mucosal epithelium stain for EpCAM (marker of proliferating cells) and CK19 (biliary marker). In addition, a novel stem cell marker, GbM, co-localizes with both EpCAM and CK19.
3. The LDA of HuAL GB1 quantifies the increase in CFU frequency that the enrichment for GbM affords. GbM⁺ cells show a 7-fold increase in CFU frequency over the GbM-cells.
4. Flow cytometric analyses of the gall bladder cells expanded in culture reveal that GbM stains a subpopulation of cells within human gall bladder. The percentage of the GbM+ sub-population in HuAL GB1 & HuAL GB3 varies considerably. More samples need to be examined to elucidate the significance of this difference.
5. The percentage of GbM+ cells in HuAL GB3 was small. A sufficient number of GbM+ cells could not be sorted for an LDA. However, GbM+ cells were sorted into culture and the CFU frequency was determined to be roughly 1/50 (the GbM⁻ sorted cells did not grow). These data are consistent with the notion that selection for GbM results in an increase in CFU frequency.

### Example 2

Rat breast ductal carcinoma cell lines were used as feeders to expand adult human and mouse gallbladder cells. Limiting Dilution Analyses (LDA) using the epithelial marker, EpCAM, were used to define the proliferative ability of the primary and cultured cells. In order to test the capacity of these cells for lineage commitment, GFP⁺ mouse gallbladder cells expanded in vitro were injected into the renal sub-capsular space of a WT mouse. GFP⁺ ductular structures were visible 4 weeks post-transplant. Furthermore, multiple clonal populations were derived from GFP⁺ mouse gallbladder cells by single cell clonogenic assays. These preliminary data are indicative of a cell population that satisfies the criteria of self-renewal and lineage commitment.

### Methods: see Example 1.

### Results:

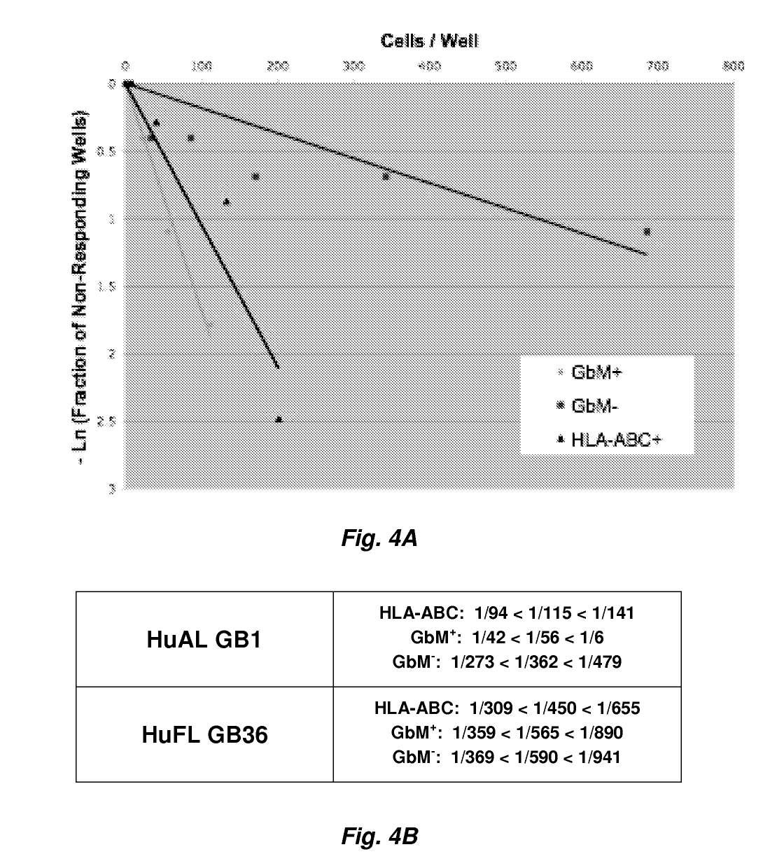

Gallbladder cells from 9 patient samples were successfully expanded in vitro. All cultures exhibited morphological heterogeneity. The hallmark of these cultures was epithelial cells growing in 2-dimensional cobblestone-like colonies (FIG. 1 let panel). Organized glandular structures, typical of cellular differentiation, smaller cells with large nuclear: cytoplasmic ratios, typical of more immature cells (FIG. 5 left panel, arrow head) and biliary type cells (arrow) were seen. In this reporting period, we examined murine cultures which were shown to lack cobblestone-like colonies, but exhibit biliary type (FIG. 5 right panel, arrow) and small cell colonies along with organotypic structures.

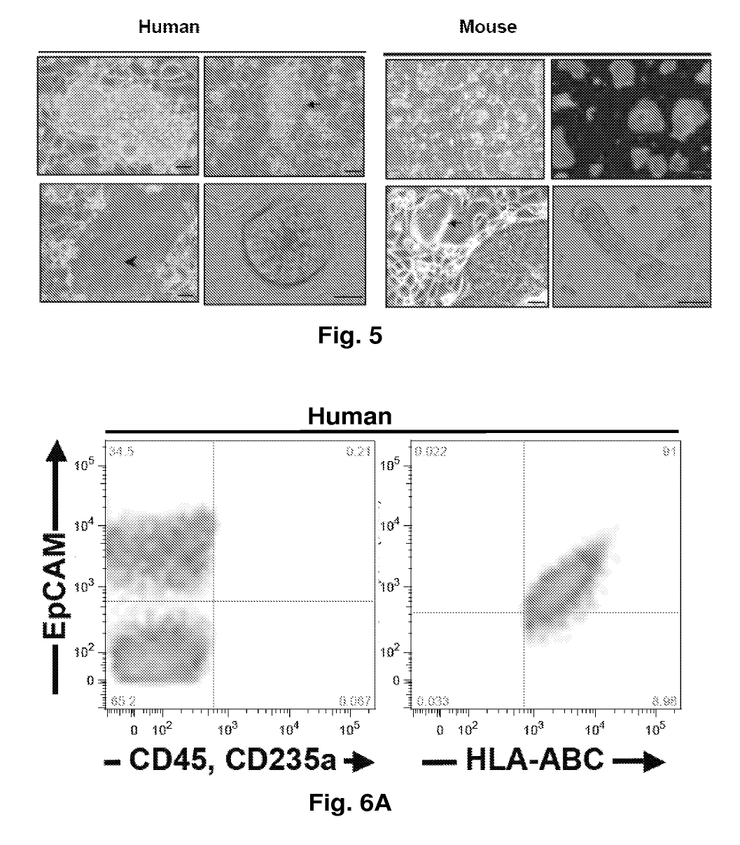

Flow Cytometric data: Flow cytometric analyses of primary and cultured human and mouse gallbladder cells indicate that only the epithelial compartment, i.e. EpCAM⁺ cells, from primary tissue, expands in culture (FIG. 6).

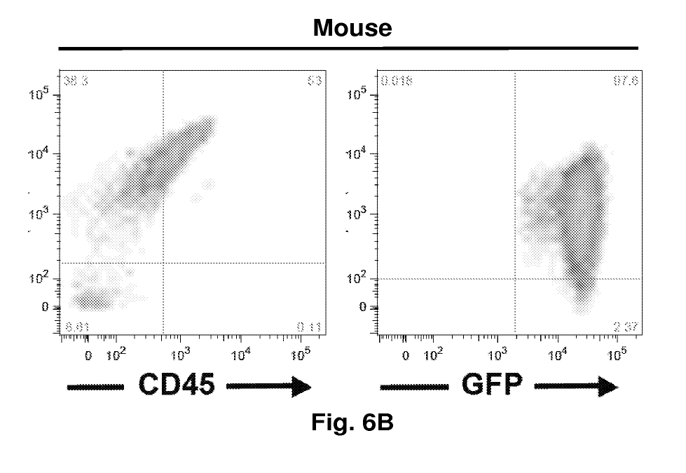

Limiting Dilution Analyses: LDAs performed on human and mouse gallbladder cells indicate enrichment in the colony forming unit (CFU) ability between primary and culture cells. These data demonstrate that the in vitro culture system supports the expansion of a more proliferative or primitive cell population (FIG. 7).

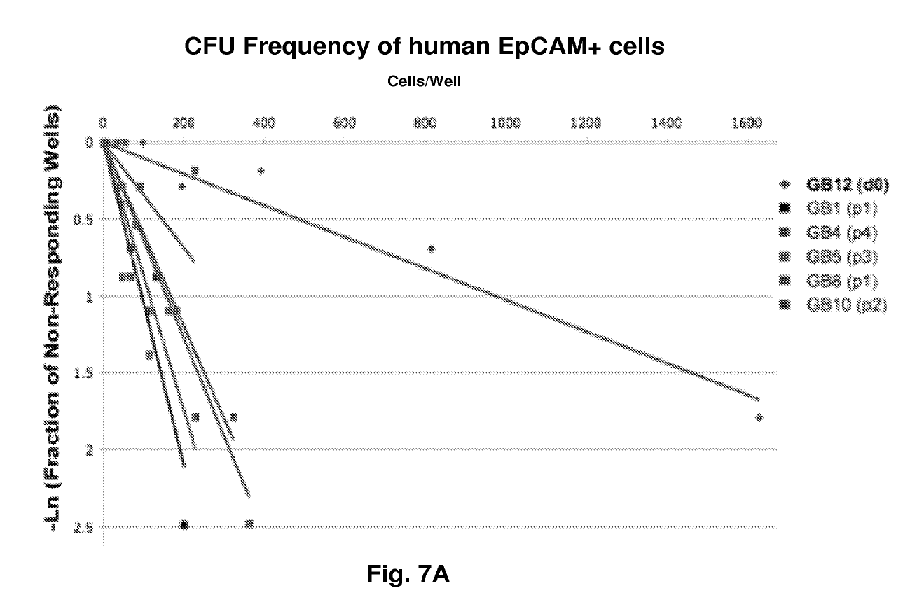

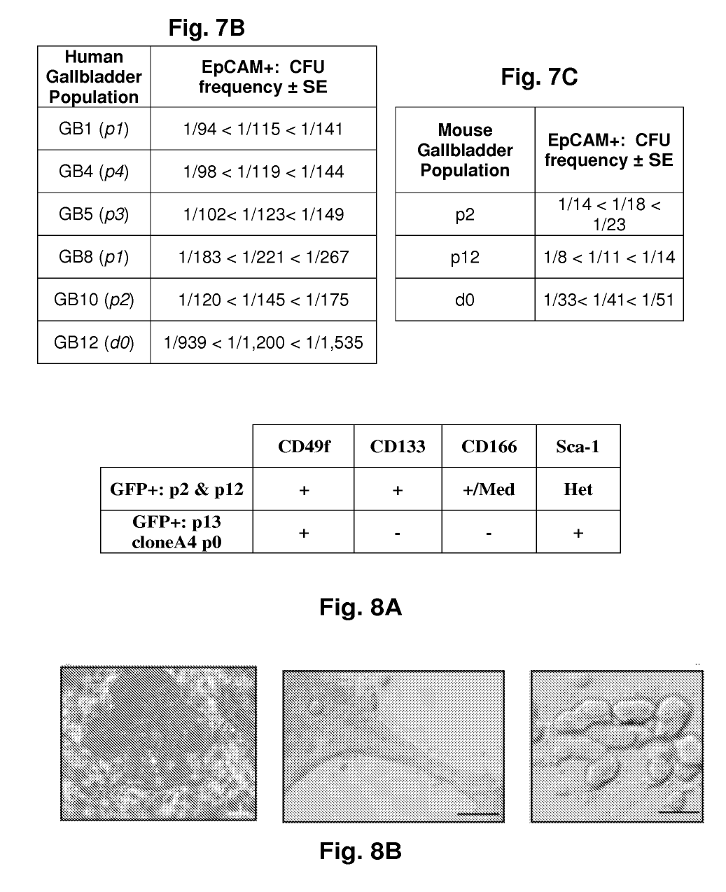

In Vivo Assay and Clonogenic Assay: Clonogenic populations were derived from GFP⁺ mouse gallbladder by single cell clonal expansion. As shown in FIG. 8, clone A4 consisted of small cell colonies (left panel), ductular structures (middle panel), and 3D organotypic structures (right panel) in culture, and exhibited a different surface marker profile than the bulk population. Furthermore, GFP⁺ mouse gallbladder formed ductular structures when injected into the renal sub-capsular space of WT mice (FIG. 9).

Summary and Future Directions: This study presents preliminary data supporting the hypothesis that a stem cell population in the adult gallbladder exists. It describes the use of a novel feeder cell layer and an in vitro colony forming unit assay in the expansion and characterization of primitive cells from adult human and mouse gallbladders. Immature/stem/precursor cells can be isolated, for example, by dilution and expansion of single cells into colonies on an irradiated cell layer and later transplanted to larger flasks.

FIG. 5 shows representative light micrographs of human and GFP⁺ mouse adult gallbladder grown on irradiated feeder cells. Human cultures contain cobblestone-like colonies of epithelial cells, billiary type cells (black arrow), smaller cells large nuclear/cytoplasmic ratios, typical of more primitive cells (arrowhead) and 3D organotypic structures. Mouse cultures lack cobblestone-like colonies, but exhibit biliary type and small cell colonies along with organotypic structures. Black scale bars=50 μM. White scale bars=100 μM.

FIGS. 2A and 6A and 6B show that only EpCAM+ cells expand on the feeder cells. FIG. 2A is a composite light micrograph showing EpCAM expression in adult human gallbladder. Expression is seen throughout the epithelia, in mucosal structures (arrowhead) and columnar epithelium lining the lumen (arrow). FIGS. 6A and 6B show flow cytometric profile of EpCAM in human and mouse adult gallbladder cells, respectively. The left plots in each panel represent non-hematopoietic cells from primary tissue. The right plots in each panel represent ABC+ human cells and GFP+ mouse cells, respectively, expanded in culture (p<10). Only EpCAM+ or epithelial cells expand in the feeder cell culture system.

FIG. 7 shows Limiting Dilution Analysis of human and mouse gallbladder cells from primary tissue and in culture on feeder cells. FIG. 7A shows regression Analysis of human gallbladder cells indicating the frequency of the colony forming unit (CFU) cells. Cell populations with steeper slope have a higher CFU frequency (FIGS. 7B and 7C) CFY frequency±SE of human and mouse gallbladder cells as determined by L-Calc®, a statistical LDA software. The feeder cell culture system selects for amore proliferative or primitive cell population in both the human and mouse gallbladder cells.

For FIG. 8, a 96-well plate was seeded with GFP+C57BL/6J mouse gallbladder cells in culture at 1 cell/well. Five different clones were obtained. FIG. 8A is a phenotypic profile of Clone A4 compared to the bulk population. FIG. 8B provides photomicrographs showing clone A4 consisted of small-cell colonies (left panel), ductular structures (middle panel), and 3D organotypic structures (right panel) in culture. Black Scale bars=50 μm, White Scale bar=100 μm.

FIG. 9 shows formation of ductular structures in vivo. In FIG. 9A, GFP⁺ mouse gallbladder cells in culture were injected into the renal sub-capsular space of WT mice. Mice were sacrificed and kidneys were removed at 2, 3 and 4 weeks post-injection. In FIG. 9B, GFP⁺ ductular structures are observed. These data evidence differentiation in vivo.

### Example 3

### Isolation of a Stem Cell Population from a Population of Gall-Bladder Derived Cells

A population of cells that are GbM⁺ are clonally expanded by first isolating a population of GbM+ cells from a population of gallbladder cells. The GbM+ cells are then diluted and plated in a 96-well culture dish with an established layer of irradiated feeder cells as described above. Cells that form colonies are then transferred to progressively larger plates, dishes or flasks having a feeder cell layer. Cells propagated in this manner may be frozen or implanted in a patient.

Since the experiments above, it has been found that although selection for GbM+/EpCAM+ cells can produce an enriched population of cells comprising progenitor cells, the progenitor cells have the phenotype EpCam+, CD133+, CD49f+, CD13−, thus enriching by affinity, cell sorting, etc. EpCam+, CD133+, CD49f+, CD13− cells from a population of gallbladder cells would effectively enrich the population of progenitor cells. For example, EpCAM+ cells may be selected by growth on an epithelial feeder layer or by affinity or cell sorting. Then one or all of CD133+, CD49f+, CD13− are used as criteria for enriching a population of progenitor cells, for example by affinity purification or FACS, or other cell sorting methods.

### Example 4

### Differentiation of Stem Cells into Hepatocytes

It is expected that the progenitor cells described herein can be differentiated into hepatocytes. In an exemplary method, human gallbladders are treated with repeated rounds of collagenases and trypsin. The cells are plated at approximately 70,000 cells/square mm on rat breast ductal carcinoma cells treated with gamma-irradiation (17,000 rads), in T25 tissue culture plates in DMEM/F12 (Fisher 10-090-CV)+0.5% FBS+1% Insulin, Transferrin, Selenium Supplement ITS) (Fisher#25-080-CR)+25 μg/ml Gentamycin (Sigma#G1397) media. The cells are allowed to attach overnight and media is removed from the plates along with unattached cells. Cold Matrigel (1 ml per 35-mm plate) is then layered on top of the cells and allowed to solidify at 37° C. for 15 min-1 h. The cells are then fed medium consisting of Dulbecco's minimum essential medium, 2 mM L-glutamine, 20 mM HEPES, 0.54 mg/l ZnCl₂, 0.75 mg/l ZnSO₄.7H₂O, 0.2 mg/l CuSO₄.5H₂O, 0.025 mg/l MnSO₄, 3 g/l glucose, 2 g/l galactose, 2 g/l BSA, 0.1 g/l ornithine, 0.03 g/l proline, 0.6 g/l nicotinamide, and 0.1 μM dexamethasone plus ITS medium supplement. This medium is modified with various concentrations of hepatocyte growth factor (HGF), epidermal growth factor (EGF), and Dexamethasone. Cells are fed three times per week with medium freshly supplemented with growth factors.

In order to test the plasticity of gallbladder cells towards hepatocytes, cultured gallbladder cells were grown in a candidate hepatic differentiation assay. The cells were grown between collagen and matrigel in the presence of HGF, EGF and Dex, at which time, they begin to express albumin mRNA.

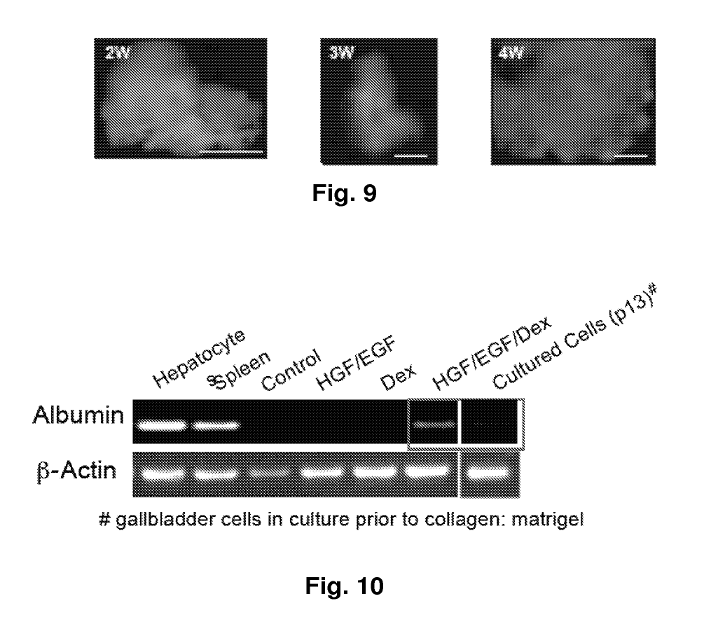

This exemplary protocol was used to differentiate the progenitor cells described herein. Briefly, cells were seeded on collagen gels and layered with matrigel essentially as described above. HGF, EGF and Dex were added to the cultures for 2 weeks, following which cells were isolated for analysis. As can be seen in an RT-PCR experiment, shown in FIG. 10, up-regulation of albumin gene expression is seen in mouse gallbladder cells treated with HGF/EGF/Dex. Control cells and cells treated with HGF/EGF, Dex and HGF/EGF/Dex expressed CK19 mRNA (data not shown). However, these cells do not engraft in or rescue Fah−/− mouse livers.

### Example 5

### In Vitro Stem Cell Assay for Gallbladder Cells

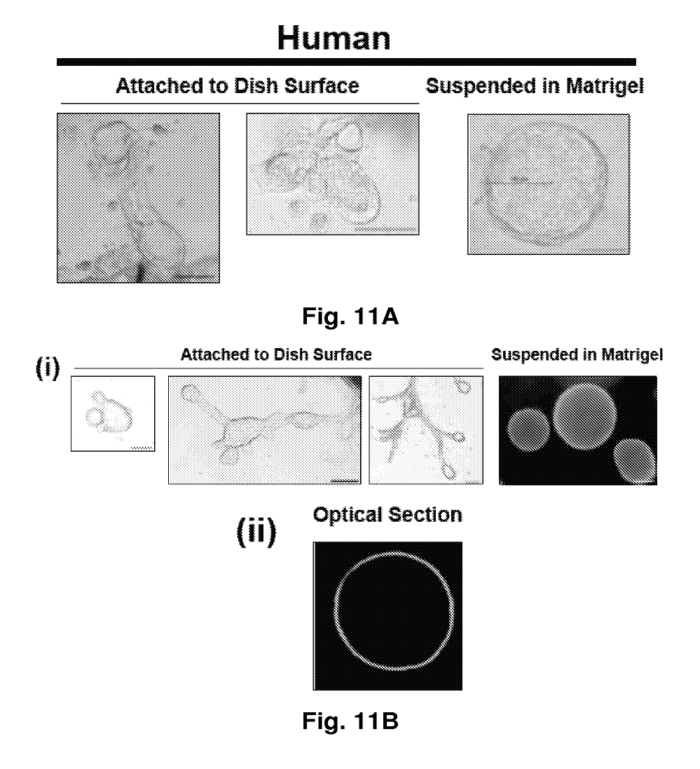

Human (<p5) and Mouse (>p5) gallbladder cells were seeded on dishes and layered with matrigel. Heterogeneous morphogenesis of the gallbladder cells was observed over ˜4 weeks. As shown in FIG. 11A, human gallbladder cells form two distinct type of structures: gallbladder-like structures with narrow lumen that are attached to the dish surface and cyst-like structures suspended in the matrigel. As shown in FIG. 11B, (i) Mouse gallbladder cells exhibit similar morphogenesis. (ii) Slice in a confocal stack of a cyst depicting a narrow lining of cells that surrounds a large lumen. Cysts expand more robustly than gallbladder structures upon passage (data not shown) and re-form morphological heterogeneity. This morphogenesis does not occur with mouse intra-hepatic bile duct cells. Scale bars=100 μm.

Whereas particular embodiments of the one or more inventions described herein have been described above for purposes of illustration, it will be evident to those skilled in the art that numerous variations of the details of those one or more inventions may be made without departing from the embodiments defined in the appended claims.

We claim:

1. A progenitor cell preparation comprising gallbladder cells enriched for EpCAM+ cells.

2. The enriched progenitor cell population of claim 1 further enriched by expanding the EpCAM+ cells in culture.

3. The enriched progenitor cell population of claim 1 comprising EpCam+, CD133+, CD49f+, CD13− cells.

4. The enriched progenitor cell population of claim 1 further enriched for CD133+, CD49f+, CD13− cells.

5. A method of preparing an enriched progenitor cell population, comprising:
a. dissociating gallbladder cells; and
b. enriching the gallbladder cells for EpCAM+ cells.

6. The method of claim 5, further comprising expanding the EpCAM+ cells in culture.

7. The method of claim 6 in which the cells are expanded on a feeder layer.

8. The method of claim 7 in which the feeder layer comprises stromal cells (e.g., fibroblasts).

9. The method of claim 7 in which the feeder layer comprises epithelial cells.

10. The method of claim 9 in which the epithelial cells are gall bladder epithelial cells.

11. The method of claim 10 in which the gall bladder epithelial cells are an immortalized cell line.

12. The method of claim 7 in which the feeder layer is irradiated to prevent growth of the feeder layer.

13. The method of claim 12 in which the feeder layer is γ-irradiated.

14. The method of claim 5, in which the gallbladder cells are enriched by binding the cells with an anti-EpCAM binding reagent and physically separating the cells that are bound to the anti-EpCAM binding reagent.

15. The method of claim 14 in which the anti-EpCAM binding reagents are attached to a substrate and the method comprises contacting the dissociated gallbladder cells are contacted with the substrate-bound binding reagents, washing unbound cells from the substrate; and eluting bound cells from the substrate.

16. The method of claim 14 in which the anti-EpCAM binding reagents are fluorescently labeled and the method comprises sorting the cells in a fluorescence activated cell sorter.

17. The method of claim 14 in which the anti-EpCam binding reagents are an antibody or a fragment thereof.

18. The method of claim 5 further comprising enriching the gallbladder cells for CD133+, CD49f+, CD13− cells.

19. A stem cell line prepared according to the method of claim 5.

20. A method of making a hepatocyte, comprising growing EpCAM+ progenitor cells enriched from human gall bladder cells in the presence of one or more of hepatocyte growth factor (HGF), epidermal growth factor (EGF), dexamethasone and transforming growth factor.
